(19) United States
(12) Patent Application Publication
(10) Pub. No.: US 2025/0152108 A1
(43) Pub. Date: May 15, 2025

(54) METHOD OF PROVIDING BIOMARKER AND DISPLAY DEVICE PERFORMING THE SAME

(71) Applicant: SAMSUNG DISPLAY CO., LTD., YONGIN-SI (KR)

(72) Inventor: CHUL KIM, Yongin-si (KR)

(21) Appl. No.: 18/731,456

(22) Filed: Jun. 3, 2024

(30) Foreign Application Priority Data
Nov. 10, 2023 (KR) 10-2023-0155849

(51) Int. Cl. A61B 5/00; A61B 5/024

(52) U.S. Cl. CPC A61B 5/742; A61B 5/02427

(57) ABSTRACT

A method of providing a biomarker on a display device includes displaying an initial guidance image that guides a user to place a finger on a sensing region of the display device. A first photoplethysmography (PPG) signal is generated by performing a PPG sensing operation when the finger is located in the sensing region. At least one tilt guidance image is displayed that requests to tilt the finger in at least one direction. At least one second PPG signal is generated by performing the PPG sensing operation when the finger is tilted in the at least one direction. An optimal PPG signal having a highest signal quality is selected among the first PPG signal and the at least one second PPG signal. The display device then displays a biomarker of the user determined based on the optimal PPG signal.

### CROSS-REFERENCE TO RELATED APPLICATION(S)

This application claims priority under 35 USC § 119 to Korean Patent Application No. 10-2023-0155849, filed on Nov. 10, 2023, in the Korean Intellectual Property Office (KIPO), the contents of which is herein incorporated by reference in its entirety.

### BACKGROUND

### 1. Field

Embodiments of the present inventive concept relate to a display device, and more particularly to a method of providing a biomarker and a display device performing the method.

### 2. Description of the Related Art

Electronic devices (e.g., a smart phone, a smart watch, etc.) have been developed that perform bio-sensing operations, e.g., a fingerprint sensing operation, a photoplethysmography (PPG) sensing operation, etc. These devices may perform the bio-sensing operations using a that is sensor separate from a display device. In this case, the size of a display region of the electronic device or the display device may be reduced and the size of a bezel may be increased.

Attempts have been made to solve this problem. For example, an in-cell light sensor technique has been used that employs an optical sensor or a light sensing pixel within the display region of the display device.

### SUMMARY

Some embodiments provide a method of providing a biomarker by a display device.

Some embodiments provide a display device providing a biomarker.

According to embodiments, there is provided a method of providing a biomarker by a display device. In the method, an initial guidance image that guides a user to place a finger on a sensing region of the display device is displayed, a first photoplethysmography (PPG) signal is generated by performing a PPG sensing operation when the finger is located on the sensing region, at least one tilt guidance image that requests the user to tilt the finger in at least one direction is displayed, at least one second PPG signal is generated by performing the PPG sensing operation when the finger is tilted in the at least one direction, an optimal PPG signal having a highest signal quality is selected among the first PPG signal and the at least one second PPG signal, and a biomarker of the user determined based on the optimal PPG signal is displayed.

In some embodiments, a display panel of the display device may include a light emitting pixel having a light emitting element, and a light sensing pixel having an organic photodiode, and the PPG sensing operation may be performed such that the light emitting pixel emits light, and the light sensing pixel senses the light reflected from a blood vessel of the finger.

In some embodiments, to display the at least one tilt guidance image, a left tilt guidance image that requests the user to tilt the finger to left may be displayed, and a right tilt guidance image that requests the user to tilt the finger to right may be displayed.

In some embodiments, to generate the at least one second PPG signal, a left tilt PPG signal may be generated by performing the PPG sensing operation when the finger is tilted to the left, and a right tilt PPG signal may be generated by performing the PPG sensing operation when the finger is tilted to the right.

In some embodiments, to select the optimal PPG signal, a magnitude of an alternate current (AC) component of the first PPG signal, a magnitude of an AC component of the left tilt PPG signal and a magnitude of an AC component of the right tilt PPG signal may be compared, and the optimal PPG signal having a largest AC component may be selected among the first PPG signal, the left tilt PPG signal and the right tilt PPG signal.

In some embodiments, to display the at least one tilt guidance image, an upward tilt guidance image that requests to tilt the finger upward may be displayed, and a downward tilt guidance image that requests to tilt the finger downward may be displayed.

In some embodiments, to generate the at least one second PPG signal, a left tilt PPG signal may be generated by performing the PPG sensing operation when the finger is tilted to the left, a right tilt PPG signal may be generated by performing the PPG sensing operation when the finger is tilted to the right, an upward tilt PPG signal may be generated by performing the PPG sensing operation when the finger is tilted upward, and a downward tilt PPG signal may be generated by performing the PPG sensing operation when the finger is tilted downward.

In some embodiments, to select the optimal PPG signal, a magnitude of an AC component of the first PPG signal, a magnitude of an AC component of the left tilt PPG signal, a magnitude of an AC component of the right tilt PPG signal, a magnitude of an AC component of the upward tilt PPG signal and a magnitude of an AC component of the downward tilt PPG signal may be compared, and the optimal PPG signal having a largest AC component may be selected among the first PPG signal, the left tilt PPG signal, the right tilt PPG signal, the upward tilt PPG signal and the downward tilt PPG signal.

In some embodiments, at least one movement guidance image that requests movement of the finger may be displayed, and at least one third PPG signal may be generated by performing the PPG sensing operation when the finger moves.

In some embodiments, to display the at least one tilt guidance image, a left tilt guidance image that requests to tilt the finger to left may be displayed, and a right tilt guidance image that requests to tilt the finger to right may be displayed. To display the at least one movement guidance image, an upward movement guidance image that requests to move the finger upward may be displayed, and a downward movement guidance image that requests to move the finger downward may be displayed.

In some embodiments, to generate the at least one second PPG signal, a left tilt PPG signal may be generated by performing the PPG sensing operation when the finger is tilted to the left, and a right tilt PPG signal may be generated by performing the PPG sensing operation when the finger is tilted to the right. To generate the at least one third PPG signal, an upward movement PPG signal may be generated by performing the PPG sensing operation when the finger moves upward, and a downward movement PPG signal may be generated by performing the PPG sensing operation when the finger moves downward.

In some embodiments, to select the optimal PPG signal, a magnitude of an AC component of the first PPG signal, a magnitude of an AC component of the left tilt PPG signal, a magnitude of an AC component of the right tilt PPG signal, a magnitude of an AC component of the upward movement PPG signal and a magnitude of an AC component of the downward movement PPG signal may be compared, and the optimal PPG signal having a largest AC component may be selected among the first PPG signal, the left tilt PPG signal, the right tilt PPG signal, the upward movement PPG signal and the downward movement PPG signal.

In some embodiments, to display the biomarker of the user, a blood pressure, a heart rate, a stress level and a cardiovascular health of the user determined based on the optimal PPG signal may be displayed.

In some embodiments, optimal guidance image information representing an optimal guidance image for the user may be stored, and the optimal guidance image may be a guidance image corresponding to the optimal PPG signal among the initial guidance image and the at least one tilt guidance image.

In some embodiments, the optimal guidance image for the user may be displayed based on the optimal guidance image information when the user subsequently requests sensing of the biomarker, a PPG signal may be generated by performing the PPG sensing operation, and the biomarker of the user determined based on the PPG signal may be displayed.

According to embodiments, there is provided a method of providing a biomarker by a display device. In the method, an initial guidance image that guides a user to place a finger on the sensing region of the display device is displayed, an initial photoplethysmography (PPG) signal is generated by performing a PPG sensing operation when the finger is located in the sensing region, a left tilt guidance image that requests to tilt the finger to left is displayed, a left tilt PPG signal is generated by performing the PPG sensing operation when the finger is tilted to the left, a right tilt guidance image that requests to tilt the finger to right is displayed, a right tilt PPG signal is generated by performing the PPG sensing operation when the finger is tilted to the right, an upward movement guidance image that requests to move the finger upward is displayed, an upward movement PPG signal is generated by performing the PPG sensing operation when the finger moves upward, a downward movement guidance image that requests to move the finger downward is displayed, a downward movement PPG signal is generated by performing the PPG sensing operation when the finger moves downward, an optimal PPG signal having a highest signal quality is selected among the initial PPG signal, the left tilt PPG signal, the right tilt PPG signal, the upward movement PPG signal and the downward movement PPG signal, and a biomarker of the user determined based on the optimal PPG signal is displayed.

In embodiments, optimal guidance image information representing an optimal guidance image for the user may be stored, and the optimal guidance image may be a guidance image corresponding to the optimal PPG signal among the initial guidance image, the left tilt guidance image, the right tilt guidance image, the upward movement guidance image and the downward movement guidance image. The optimal guidance image for the user may be displayed based on the optimal guidance image information when the user subsequently requests sensing of the biomarker.

According to embodiments, there is provided a display device including a display panel including a plurality of pixel groups, and a panel driver configured to drive the display panel. Each of the pixel groups includes a light emitting pixel having a light emitting element, and a light sensing pixel having an organic photodiode. The panel driver drives the display panel to display an initial guidance image that guides a user to place a finger on a sensing region of the display panel, generates a first photoplethysmography (PPG) signal by performing a PPG sensing operation when the finger is located in the sensing region, drives the display panel to display at least one tilt guidance image that requests to tilt the finger in at least one direction, generates at least one second PPG signal by performing the PPG sensing operation when the finger is tilted in the at least one direction, selects an optimal PPG signal having a highest signal quality among the first PPG signal and the at least one second PPG signal, and drives the display panel to display a biomarker of the user determined based on the optimal PPG signal.

In embodiments, to perform the PPG sensing operation, the panel driver may drive the light emitting pixels of the pixel groups located in an adjacent region that is adjacent to the sensing region to emit light, and may drive the light sensing pixels of the pixel groups located in the sensing region to sense the light reflected from a blood vessel of the finger.

In embodiments, the panel driver may store optimal guidance image information representing an optimal guidance image for the user, and the optimal guidance image may be a guidance image corresponding to the optimal PPG signal among the initial guidance image and the at least one tilt guidance image. The panel driver may display the optimal guidance image for the user based on the optimal guidance image information when the user subsequently requests sensing of the biomarker.

As described above, in a method of providing a biomarker and a display device according to embodiments, a first photoplethysmography (PPG) signal may be generated by performing a PPG sensing operation when a finger of a user is located in a sensing region of the display device, at least one second PPG signal may be generated by performing the PPG sensing operation after at least one tilt guidance image is displayed, an optimal PPG signal may be selected among the first PPG signal and the at least one second PPG signal, and a biomarker of the user may be determined based on the optimal PPG signal. Accordingly, a contact surface between the finger and the sensing region may be optimized for each user, a signal quality of a PPG signal may be improved, and the biomarker of the user may be accurately sensed.

In accordance with one or more embodiments, a method of providing a biomarker by a display device includes generating a first biological signal based on a sensing operation performed for a finger of a user on a display screen; displaying a finger adjustment image instructing the user to adjust a position of the finger on the display screen; generating a second biological signal based on a sensing operation performed for the adjusted position of the finger; selecting the first biological signal or the second biological signal; and displaying a biomarker on the display screen based on the selected first biological signal or the second biological signal.

Each of the first biological signal and the second biological signal may be a photoplethysmography (PPG) signal. Selecting the first biological signal or the second biological signal may include determining a signal quality of the first PPG signal; determining a signal quality of the second PPG signal; comparing the signal quality of the first PPG signal and the signal quality of the second PPG signal; and selecting the second PPG signal based on the comparison. The second PPG signal may have a higher signal quality than the first PPG signal. The finger adjustment image may instruct the user to tilt or move the finger.

### BRIEF DESCRIPTION OF THE DRAWINGS

Illustrative, non-limiting embodiments will be more clearly understood from the following detailed description in conjunction with the accompanying drawings.

FIG. 1 is a flowchart illustrating a method of providing a biomarker by a display device according to embodiments.

FIG. 2 is a diagram illustrating an example of an initial guidance image displayed on a display device.

FIG. 3 is a diagram illustrating an example of a display device that performs a photoplethysmography (PPG) sensing operation.

FIG. 4 is a diagram illustrating examples of blood vessel distributions of fingers of a plurality of users.

FIG. 5A is a diagram illustrating an example of a left tilt guidance image displayed on a display device, and FIG. 5B is a diagram illustrating an example of a finger tilted to the left.

FIG. 6A is a diagram illustrating an example of a right tilt guidance image displayed on a display device, and FIG. 6B is a diagram illustrating an example of a finger tilted to the right.

FIG. 7A is a diagram illustrating an example of an upward movement guidance image displayed on a display device, and FIG. 7B is a diagram illustrating an example of a finger moving upward.

FIG. 8A is a diagram illustrating an example of a downward movement guidance image displayed on a display device, and FIG. 8B is a diagram illustrating an example of a finger moving downward.

FIG. 9 is a diagram for describing an example in which an optimal PPG signal is selected from among a plurality of PPG signals.

FIG. 10 is a diagram illustrating an example of a biomarker displayed on a display device.

FIG. 11 is a diagram illustrating an example of an image displayed on a display device while a PPG sensing operation is performed.

FIG. 12 is a flowchart illustrating a method of providing a biomarker by a display device according to embodiments.

FIG. 13A is a diagram illustrating an example of an upward tilt guidance image displayed on a display device, and FIG. 13B is a diagram illustrating an example of a finger tilted upward.

FIG. 14A is a diagram illustrating an example of a downward tilt guidance image displayed on a display device, and FIG. 14B is a diagram illustrating an example of a finger tilted downward.

FIG. 15 is a block diagram illustrating a display device according to embodiments.

FIG. 16 is a circuit diagram illustrating an example of a light emitting pixel and a light sensing pixel included in a display device according to embodiments.

FIG. 17 is a diagram illustrating an example of a sensing region in which a light sensing pixel is driven and an adjacent region in which a light emitting pixel is driven.

FIG. 18 is a block diagram illustrating an electronic device including a display device according to embodiments.

### DETAILED DESCRIPTION OF THE EMBODIMENTS

Hereinafter, embodiments of the present inventive concept will be explained in detail with reference to the accompanying drawings.

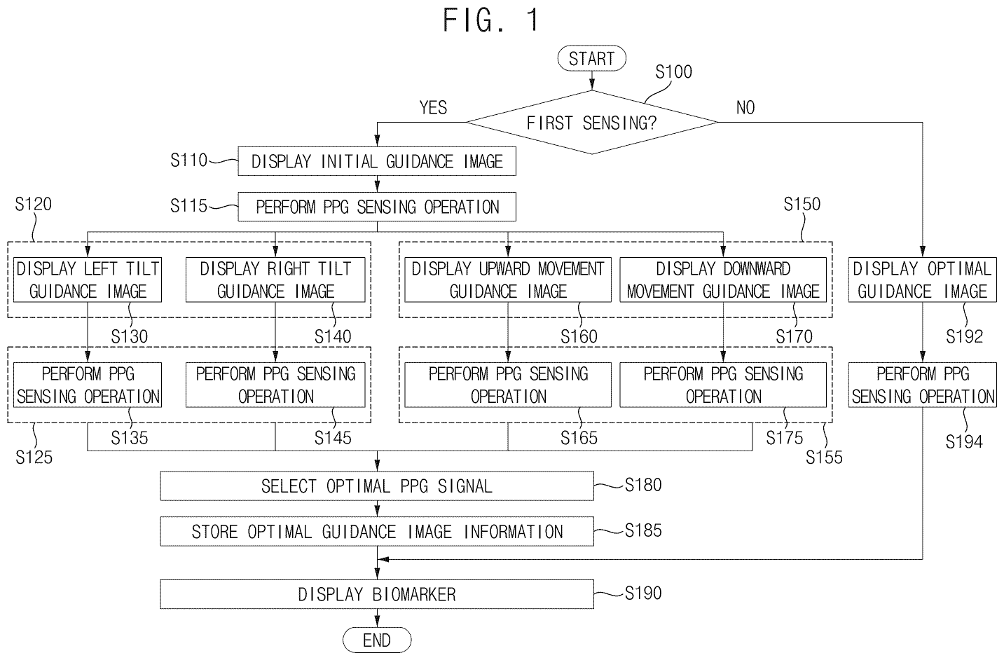

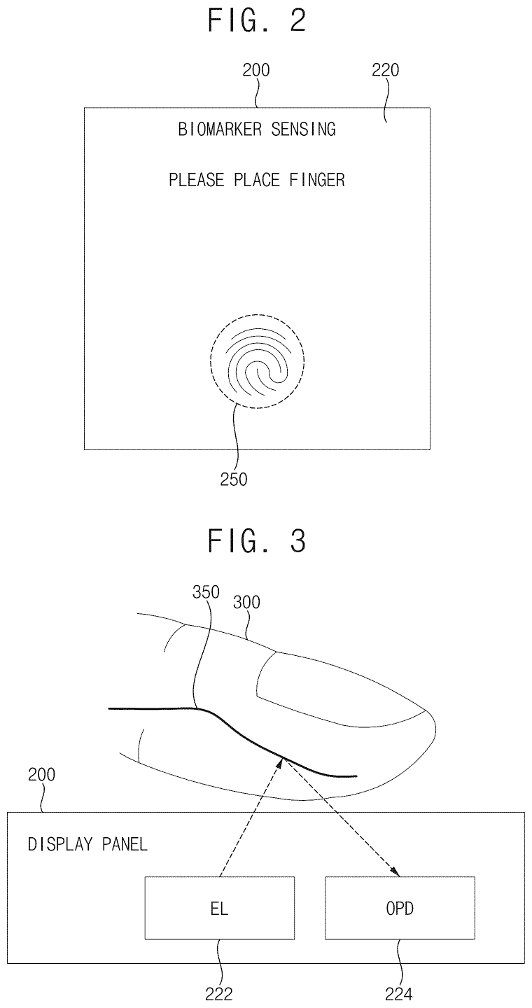

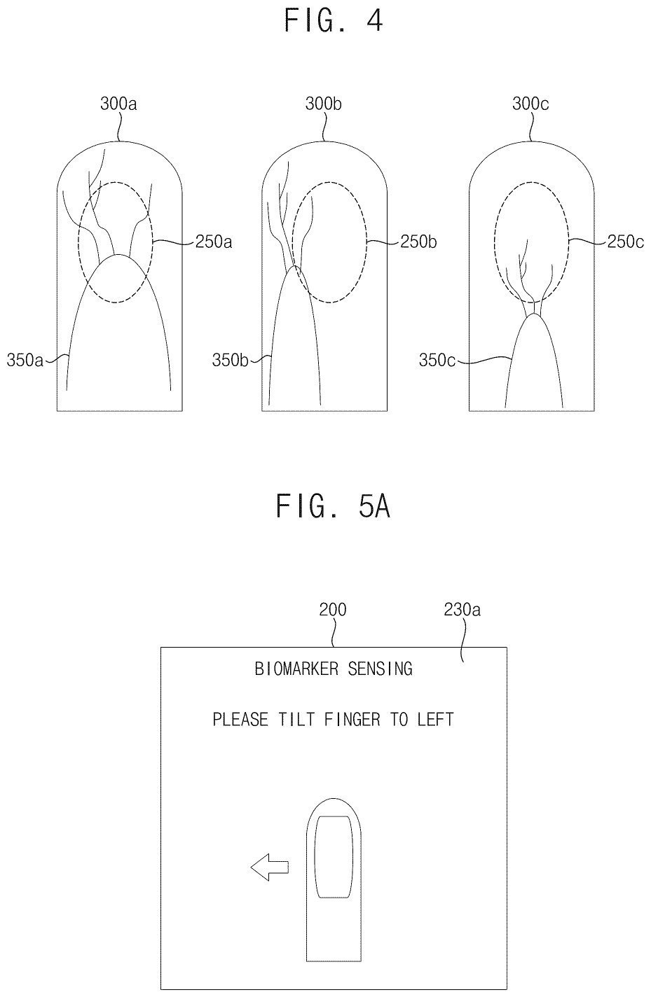

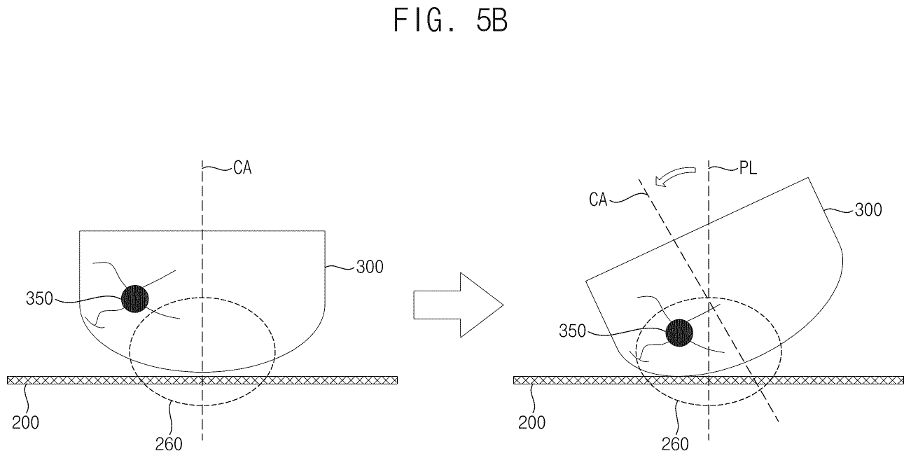

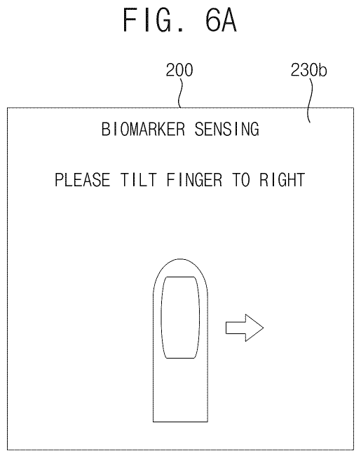

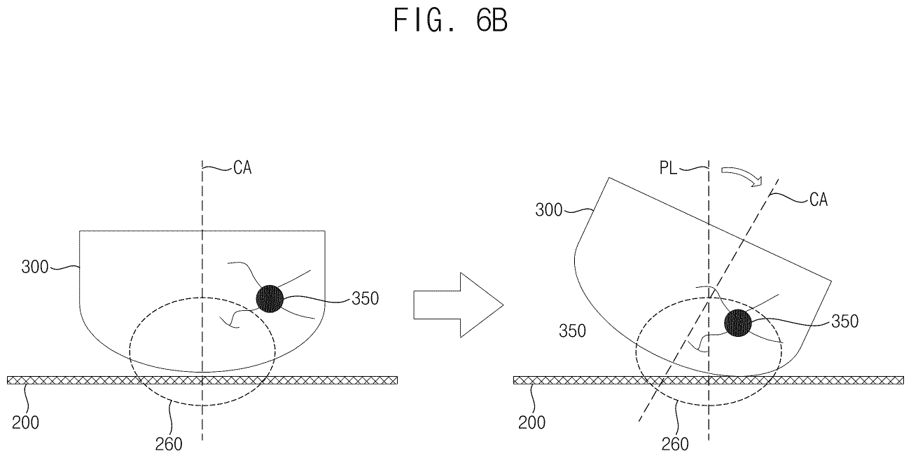

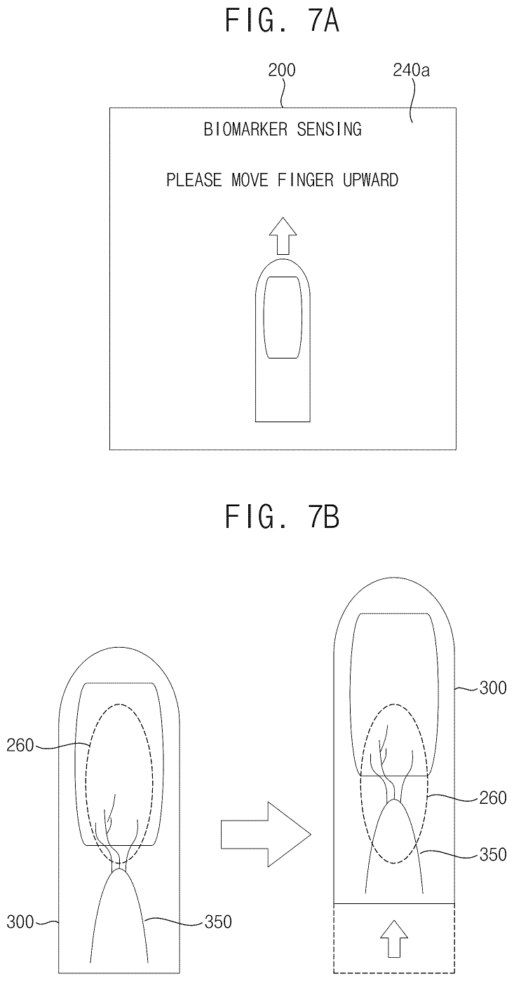

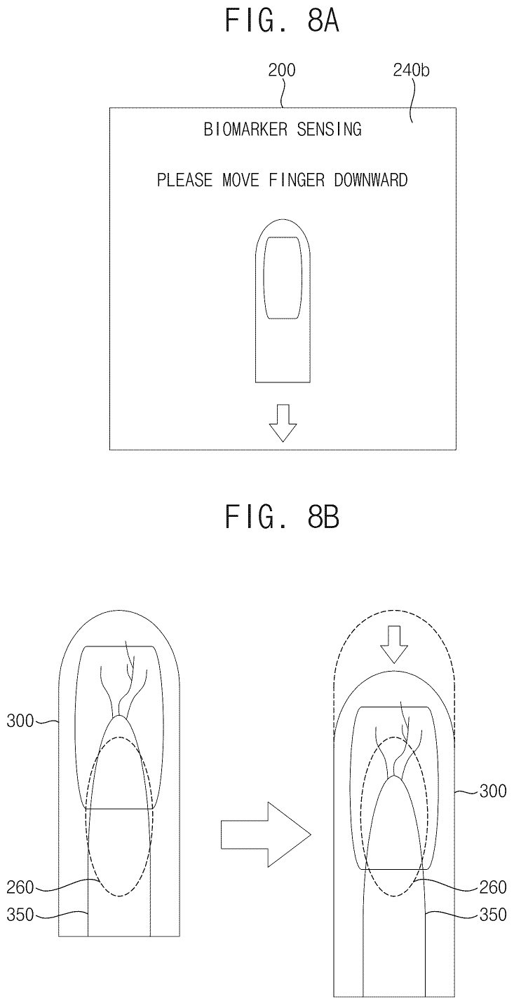

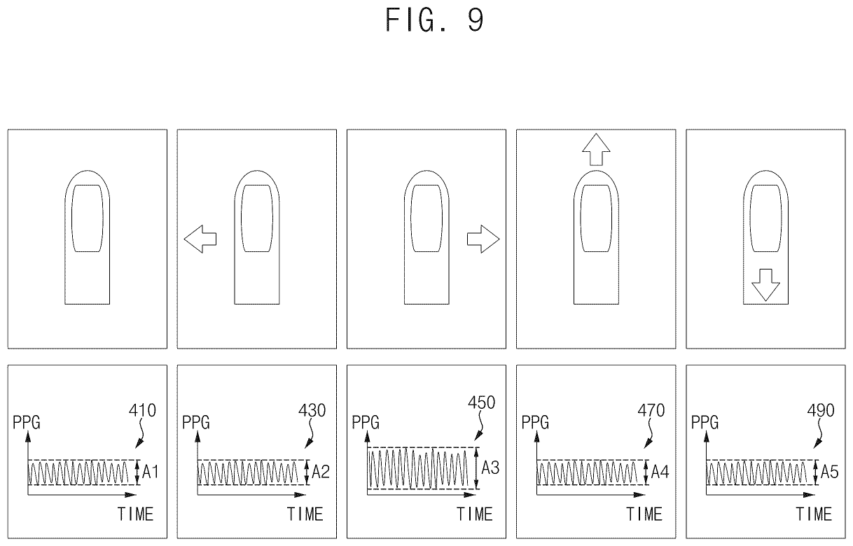

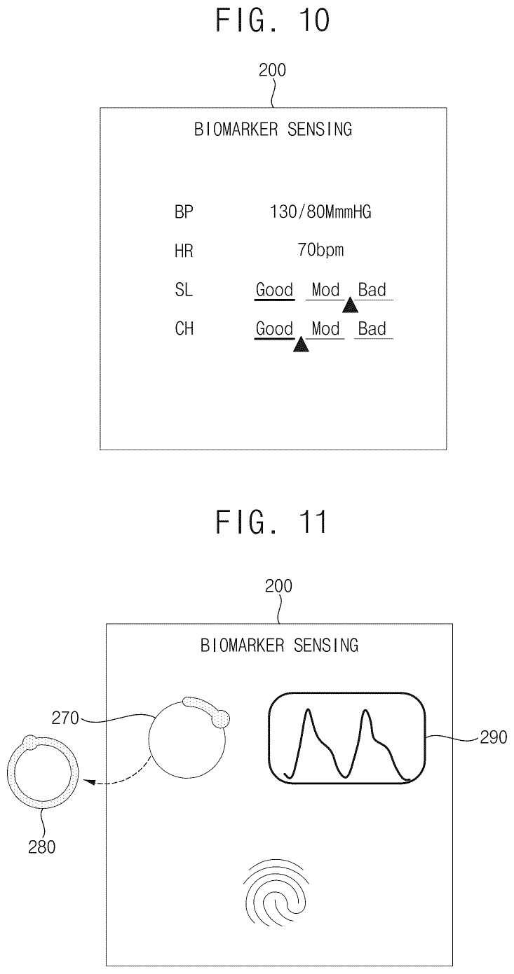

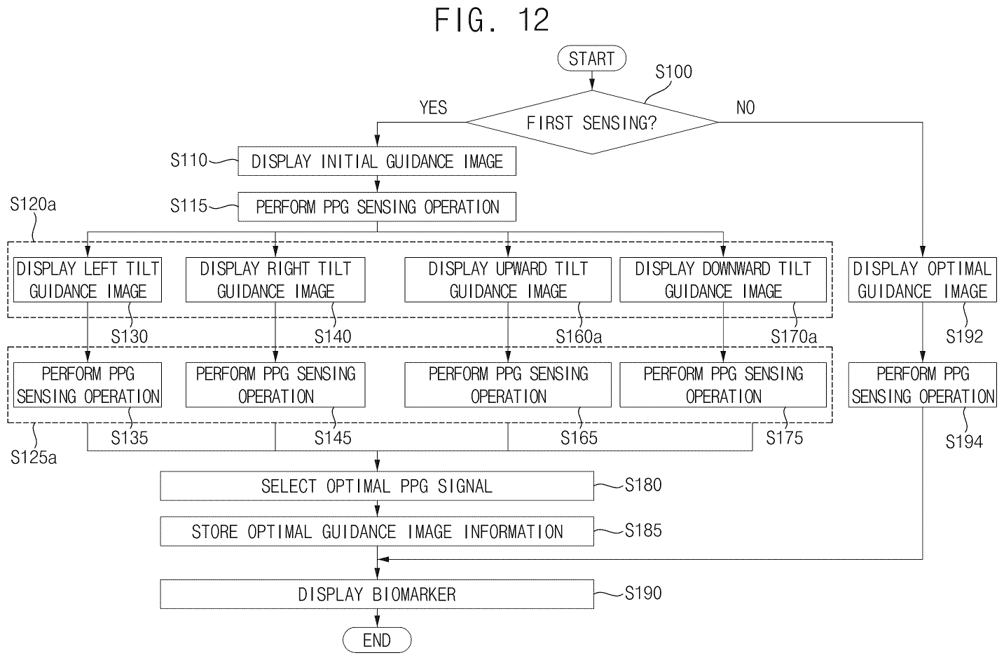

FIG. 1 is a flowchart illustrating a method of providing a biomarker by a display device according to embodiments, FIG. 2 is a diagram illustrating an example of an initial guidance image displayed on a display device, FIG. 3 is a diagram illustrating an example of a display device that performs a photoplethysmography (PPG) sensing operation, FIG. 4 is a diagram illustrating examples of blood vessel distributions of fingers of a plurality of users, FIG. 5A is a diagram illustrating an example of a left tilt guidance image displayed on a display device, FIG. 5B is a diagram illustrating an example of a finger tilted to the left, FIG. 6A is a diagram illustrating an example of a right tilt guidance image displayed on a display device, FIG. 6B is a diagram illustrating an example of a finger tilted to the right, FIG. 7A is a diagram illustrating an example of an upward movement guidance image displayed on a display device, FIG. 7B is a diagram illustrating an example of a finger moving upward, FIG. 8A is a diagram illustrating an example of a downward movement guidance image displayed on a display device, FIG. 8B is a diagram illustrating an example of a finger moving downward, FIG. 9 is a diagram for describing an example in which an optimal PPG signal is selected from among a plurality of PPG signals, FIG. 10 is a diagram illustrating an example of a biomarker displayed on a display device, and FIG. 11 is a diagram illustrating an example of an image displayed on a display device while a PPG sensing operation is performed.

Referring to FIGS. 1 and 2, the method of providing a biomarker by a display device 200 according to embodiments includes as an initial operation determining whether a first biomarker for a user is to be sensed. When a first biomarker sensing for the user is performed (S100: YES), the display device 200 may display an initial guidance image 220 (S110). For example, as illustrated in FIG. 2, the display device 200 may display the initial guidance image 220 that guides the user to place a finger on a sensing region 250 of the display screen. Further, in some embodiments, to guide the location of the sensing region 250, the initial guidance image 220 may include an icon or other guidance indicia, such as but not limited to a fingerprint image, within the sensing region 250 as illustrated in FIG. 2, but is not limited thereto.

When the finger of the user is located in the sensing region 250, a processor of the display device 200 may perform a photoplethysmography (PPG) sensing operation to generate a first PPG signal (S115). For example, as illustrated in FIG. 3, a display panel of the display device 200 may include a light emitting pixel 222 having a light emitting element EL, and a light sensing pixel 224 having an organic photodiode OPD. The PPG sensing operation may be performed such that the light emitting pixel 222 may emit light, and the light sensing pixel 224 may sense the light reflected from a blood vessel 350 of the finger 300 of the user. In another embodiment, the processor of the display device 200 may generate another type of biological signal, e.g., a biological signal different from a PPG signal.

For example, when the heart of the user contracts and the volume of the blood vessel 350 increases, the amount of hemoglobin in the blood vessel 350 may increase, a light intensity absorbed by the hemoglobin may increase, and the light sensing pixel 224 may measure a relatively low light intensity of reflected light. In contrast, when the heart of the user expands (or relaxes) and the volume of the blood vessel 350 decreases, the amount of hemoglobin in the blood vessel 350 may decrease, the light intensity absorbed by the hemoglobin may decrease, and the light sensing pixel 224 may measure a relatively high light intensity of the reflected light. The display device 200 may generate a PPG signal indicating the volume of the blood vessel 350 based on the light intensity measured by the light sensing pixel 224.

Meanwhile, the distributions of blood vessels 350 in the fingers 300 of a plurality of users may be different from each other. As a result, the first PPG signals among the plurality of users may vary in terms of quality. For example, the first PPG signal generated when the finger 300 has an initial position may have a low signal quality depending on the user. Here, the signal quality of the PPG signal may be determined in a variety of ways. For example, the signal quality of the PPG signal may be determined by a signal quality index (SQI) of the PPG signal, may be determined by a signal-to-noise ratio (SNR) of the PPG signal, and/or may be determined by an amount of an alternate current (AC) component of the PPG signal. For example, as illustrated in FIG. 4, in a case where a blood vessel 350a of a finger 300a of a first user is distributed corresponding to the sensing region 250a, the first PPG signal for the first user may have a relatively high signal quality, e.g., greater than a predetermined value. For example, a relatively high signal quality may be an SQI greater than a predetermined value, an SNR greater than a predetermined value, or an AC component greater than a predetermined value.

However, in a case where a blood vessel 350b of a finger 300b of a second user is biased to the left with respect to the sensing region 250b, or in a case where a blood vessel 350c of a finger 300c of a third user is biased downward with respect to the sensing region 250c, the first PPG signal for the second user or the first PPG signal for the third user may have a relatively low signal quality, e.g., lower than any of the aforementioned predetermined values. In this case, a biomarker determined based on the first PPG signal for the second user or the first PPG signal for the third user may not always prove to be accurate.

To prevent deterioration in the signal quality of the PPG signal (which may arise as a result of the distribution (or positioning) of the blood vessel 350 within the finger 300), the display device 200 according to embodiments may display at least one tilt guidance image that requests the user to tilt his finger 300 in at least one direction (S120), and may additionally generate at least one second PPG signal by performing the PPG sensing operation when the finger 300 is tilted in the at least one direction (S125).

In some embodiments, as illustrated in FIG. 5A, the display device 200 may display a left tilt guidance image 230a that requests the user to tilt his finger 300 to the left (S130), in order to bring blood vessel 350 into an improved sensing space. FIG. 5B is a horizontal view illustrating an example where the user tilts the finger 300 to the left. When the user views the left tilt guidance image 230a, as illustrated in FIG. 5B, the user may tilt his finger 300 to the left such that a central axis CA of the finger 300 is tilted to the left from a vertical line PL perpendicular to the display device 200 in the horizontal view. When the finger 300 is tilted to the left, the display device 200 may perform the PPG sensing operation again to generate a left tilt PPG signal (S135). Here, the left tilt PPG signal may be a PPG signal generated by the PPG sensing operation after the finger 300 is tilted to the left.

As illustrated in FIG. 5B, in the initial position of the finger, most if not all of blood vessel 350 is not in the sensing space 260 of the sensing region 250 of FIG. 2. As a result, the first PPG signal may have low signal quality. Here, the sensing region 250 may refer to a region of the display panel of the display device 200 in which the light sensing pixel 224 is driven to sense light reflected from the blood vessel 350 of the finger 300, and the sensing space 260 may refer to a space within the finger 300 that is able to be sensed by the sensing region 250 of the display device 200. On the other hand, when the blood vessel 350 of the finger 300 of the user is biased to the left, most if not all of the blood vessel 350 of the finger 300 may exist within the sensing space 260 that is to be sensed in the sensing region 250. Thus, tilting of the finger 300 of the user may generate a left tilt PPG signal that has high signal quality.

Further, as illustrated in FIG. 6A, the display device 200 may display a right tilt guidance image 230b that requests to tilt the finger 300 to the right (S140). FIG. 6B is a horizontal view illustrating an example where the user tilts the finger 300 to the right. When the user views the right tilt guidance image 230b, as illustrated in FIG. 6B, the user may tilt the finger 300 to the right such that the central axis CA of the finger 300 is tilted to the right from the vertical line PL perpendicular to the display device 200 in the horizontal view. When the finger 300 is tilted to the right, the display device 200 may again perform the PPG sensing operation to generate a right tilt PPG signal (S145). Here, the right tilt PPG signal may refer to a PPG signal generated by the PPG sensing operation after the finger 300 is tilted to the right.

As illustrated in FIG. 6B, when the finger 300 is in an initial position, there may be almost no portion of the blood vessel 350 within the sensing space 260. As a result, the first PPG signal may have low signal quality. However, when the finger 300 is tilted to the right, a greater portion (or even most) of the blood vessel 350 of the finger 300 may exist within the sensing space 260. Thus, the right tilt PPG signal may have a relatively high signal quality. In one embodiment, only one of the left tilt guidance image or the right tilt guidance image may be displayed, with an attendant PPG sensing operation performed. In another embodiment, both of the left tilt guidance image and the right tilt guidance image may be displayed, with attendant PPG sensing operations performed. In one embodiment, when a PPG signal with low quality is generated when the finger is in the initial position and a left tilt position, the right tilt sensing operation may be performed in order to obtain a high quality PPG signal. In one embodiment, when a PPG signal of low quality is generated when the finger is in the initial position and the left tilt position, the left tilt sensing operation may be performed in order to obtain a high quality PPG signal. In one embodiment, both of the left tilt sensing operation and the right tilt sensing operation may always be performed. In accordance with one or more embodiments, an upward tilt sensing operation and a downward tilt sensing operation may also be performed in response to corresponding image tilt images, in order to generate corresponding PPG signals.

Further, the display device 200 according to embodiments may display at least one movement guidance image that requests the user to move the finger 300 (S150). When the finger 300 moves, the display device 200 may additionally generate at least one third PPG signal by performing the PPG sensing operation (S155).

In some embodiments, as illustrated in FIG. 7A, the display device 200 may display an upward movement guidance image 240a that requests the user to move his finger 300 upward (S160). FIG. 7B is a top view illustrating an example where the user moves the finger 300 upward. When the user views the upward movement guidance image 240a, as illustrated in FIG. 7B, the user may move the finger 300 upward. When the finger 300 moves upward, the display device 200 may perform the PPG sensing operation to generate an upward movement PPG signal (S165). Here, the upward movement PPG signal may refer to a PPG signal generated by the PPG sensing operation after the finger 300 moves upward.

As illustrated in FIG. 7B, when the finger 300 of the user is in an initial position (e.g., biased in a downward position before the upward movement), there may be almost no portion of the blood vessel 350 within the sensing space 260. As a result, the first PPG signal may have low signal quality, e.g., below a predetermined value. However, in this case, when the finger 300 moves upward in response to the upward movement guidance image 240a, a greater portion (or most) of the blood vessel 350 of the finger 300 may exist within the sensing space 260. Thus, the upward movement PPG signal may have high signal quality, e.g., above the predetermined value.

Further, as illustrated in FIG. 8A, the display device 200 may display a downward movement guidance image 240b that requests to move the finger 300 downward (S170). FIG. 8B is a top view illustrating an example where the user moves his finger 300 downward. When the user views the downward movement guidance image 240b, as illustrated in FIG. 8B, the user may move his finger 300 downward. When the user's finger 300 moves downward, the display device 200 may perform the PPG sensing operation to generate a downward movement PPG signal (S175).

Here, the downward movement PPG signal may refer to a PPG signal generated by the PPG sensing operation after the finger 300 moves downward. As illustrated in FIG. 8B, in a case where the blood vessel 350 of the finger 300 of the user is an initial position (e.g., biased upward), there may be almost no portion of blood vessel 350 within the sensing space 260. Thus the first PPG signal may have low signal quality. However, in this case, when the finger 300 moves downward, a greater portion (or most) of the blood vessel 350 of the finger 300 may exist within the sensing space 260. Thus, the downward movement PPG signal may have high signal quality.

In one embodiment, only one of the upward movement PPG signal or the downward movement PPG signal may be generated. In other embodiments, both of the upward PPG signal and the downward PPG signal may be generated. For example, when one of the upward movement PPG signal or the downward movement PPG signal is generated to have low signal quality (e.g., below a predetermined value), the other of the upward movement PPG signal or the downward movement PPG signal may be generated.

In one embodiment, the display device 200 may select an optimal PPG signal having the highest signal quality among the first PPG signal, the at least one second PPG signal and/or the at least one third PPG signal (S180). In some embodiments, the display device 200 may select a PPG signal having the largest AC component among the first, second and third PPG signals as the optimal PPG signal. In other embodiments, the display device 200 may select a PPG signal having the highest SQI among the first, second and third PPG signals as the optimal PPG signal. In still other embodiments, the display device 200 may select a PPG signal having the highest SNR among the first, second and third PPG signals as the optimal PPG signal.

In FIG. 9, 410 may represent the first PPG signal generated when the finger 300 has the initial position, 430 may represent the left tilt PPG signal generated when the finger 300 is tilted to the left, 450 may represent the right tilt PPG signal generated when the finger 300 is tilted to the right, 470 may represent the upward movement PPG signal generated when the finger 300 moves upward, and 490 may represent the downward movement PPG signal generated when the finger 300 moves downward. In the example illustrated in FIG. 9, the display device 200 may compare an amount A1 of an AC component of the first PPG signal 410, an amount A2 of an AC component of the left tilt PPG signal 430, an amount A3 of an AC component of the right tilt PPG signal 450, an amount A4 of an AC component of the upward movement PPG signal 470 and an amount A5 of an AC component of the downward movement PPG signal 490 with one another.

The amount of the AC component may be determined, for example, by calculating a difference between a maximum value and a minimum value of each PPG signal, but is not limited thereto. Further, the display device 200 may select the optimal PPG signal having the largest AC component among the first PPG signal 410, the left tilt PPG signal 430, the right tilt PPG signal 450, the upward movement PPG signal 470 and the downward movement PPG signal 490. That is, in the example illustrated in FIG. 9, since the AC component of the right tilt PPG signal 450 has the largest amount A3, the display device 200 may select the right tilt PPG signal 450 as the optimal PPG signal.

The display device 200 may store optimal guidance image information representing an optimal guidance image (S185). Here the optimal guidance image may be a guidance image corresponding to the optimal PPG signal among the initial guidance image, the at least one tilt guidance image and the at least one movement guidance image. For example, as illustrated in FIG. 9, in a case where the right tilt PPG signal 450 is selected as the optimal PPG signal, the display device 200 may store the optimal guidance image information representing the right tilt guidance image 230b as the optimal guidance image.

Further, the display device 200 may determine a biomarker of the user based on the optimal PPG signal and may display the biomarker (S190). In some embodiments, as illustrated in FIG. 10, the display device 200 may display, as the biomarker, a blood pressure BP, a heart rate HR, a stress level SL and/or a cardiovascular health CH of the user. For example, the blood pressure BP may be determined by detecting a feature of the optimal PPG signal and by performing an analysis (e.g., machine learning, neural network analysis, etc.) on the feature of the optimal PPG signal. The heart rate HR may be determined based on a period of the optimal PPG signal. The stress level SL may be determined based on a change of the period of the optimal PPG signal. The cardiovascular health CH may be determined based on a crest time of the optimal PPG signal, or a difference in blood pressure between left and right fingers. In other embodiments, the display device 200 may further display, as the biomarker, a respiratory rate, a blood vessel age (or a blood vessel elasticity), and/or an oxygen saturation. For example, the breathing rate may be determined based on a period of a low frequency component of the optimal PPG signal, the blood vessel age may be determined based on a wave shape of the optimal PPG signal, and the oxygen saturation may be determined by an intensity difference between green reflected light and red reflected light.

The display of at least one tilt guidance image 230a and 230b and at least one movement guidance image 240a and 240b, the selection of the optimal PPG signal and the storage of the optimal guidance image information may be performed, for example, when the first biomarker sensing for the user is performed. If the user subsequently requests sensing of the biomarker after the optimal guidance image information is stored (S100: NO), the display device 200 may display the optimal guidance image for the user based on the optimal guidance image information (S192). For example, in a case where the optimal guidance image information representing the right tilt guidance image 230b is stored, the display device 200 may display the right tilt guidance image 230b in response to the subsequent request. When the user views the optimal guidance image and places the finger 300 at an optimal position, the display device 200 may generate a PPG signal by performing the PPG sensing operation (S194). While the PPG sensing operation is performed, as illustrated in FIG. 11, the display device 200 may display a progress image 270 representing a progress status of the PPG sensing operation, and/or may display a real-time image 290 representing a PPG signal generated by the PPG sensing operation. Until the PPG sensing operation is completed, the progress image 270 may gradually change to a completion image 280.

As described above, in the method of providing the biomarker according to embodiments, the first PPG signal 410 may be generated by performing the PPG sensing operation when the finger 300 of the user is located in the sensing region 250 of the display device 200. At least one second PPG signal 430 or 450 may be generated by performing the PPG sensing operation after at least one tilt guidance image 230a and 230b is displayed. At least one third PPG signal 470 or 490 may be generated by performing the PPG sensing operation after at least one movement guidance image 240a and 240b is displayed. The optimal PPG signal may be selected among the first PPG signal 410, the at least one second PPG signal 430 and 450 and the at least one third PPG signal 470 and 490, and the biomarker of the user may be determined based on the optimal PPG signal. Accordingly, a contact surface between the finger 300 and the sensing region 250 may be optimized for each user, the signal quality of the PPG signal may be improved, and the biomarker of the user may be accurately sensed.

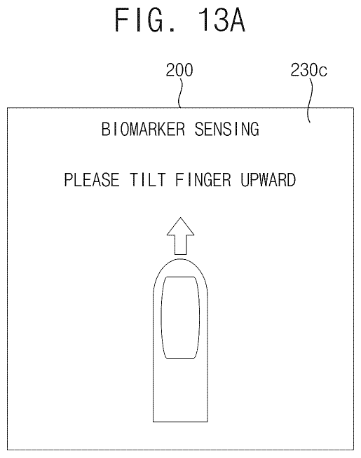

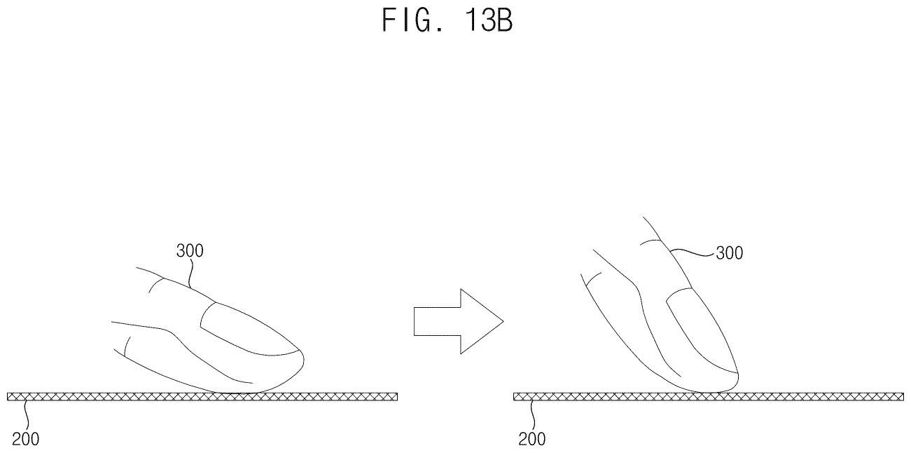

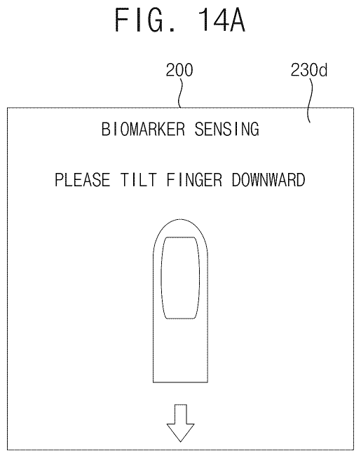

FIG. 12 is a flowchart illustrating a method of providing a biomarker by a display device according to embodiments, FIG. 13A is a diagram illustrating an example of an upward tilt guidance image displayed on a display device, FIG. 13B is a diagram illustrating an example of a finger tilted upward, FIG. 14A is a diagram illustrating an example of a downward tilt guidance image displayed on a display device, and FIG. 14B is a diagram illustrating an example of a finger tilted downward.

Referring to FIGS. 2 and 12, in a method of providing a biomarker by a display device 200 according to embodiments, when a first biomarker sensing operation for a user is performed (S100: YES), the display device 200 may display an initial guidance image 220 (S110). When a finger of the user is located in the sensing region 250, the display device 200 may perform a PPG sensing operation to generate a first PPG signal (S115).

The display device 200 may display at least one tilt guidance image that requests the user to tilt his finger in at least one direction (S120), and may additionally generate at least one second PPG signal by performing the PPG sensing operation when the finger is tilted in the at least one direction (S125). The display device 200 may display a left tilt guidance image that requests the user to tilt his finger to the left (S130), may perform the PPG sensing operation to generate a left tilt PPG signal (S135), may display a right tilt guidance image that requests the user to tilt his finger to the right (S140), and may perform the PPG sensing operation to generate a right tilt PPG signal (S145).

In some embodiments, as illustrated in FIG. 13A, the display device 200 may further display an upward tilt guidance image 230c that requests the user to tilt his finger upward (S160a). When the user views the upward tilt guidance image 230c, the user may tilt the finger 300 upward (or forward) as illustrated in FIG. 13B. When the finger 300 is tilted upward, the display device 200 may perform the PPG sensing operation to generate an upward tilt PPG signal (S165). Here, the upward tilt PPG signal may refer to a PPG signal generated by the PPG sensing operation after the finger 300 is tilted upward (or forward).

Further, as illustrated in FIG. 14A, the display device 200 may further display a downward tilt guidance image 230d that requests to tilt the finger downward (S170a). When the user views the downward tilt guidance image 230d, the user may tilt his finger downward (or backward) as illustrated in FIG. 14B. When the finger 300 is tilted downward, the display device 200 may perform the PPG sensing operation to generate a downward tilt PPG signal (S175). Here, the downward tilt PPG signal may refer to a PPG signal generated by the PPG sensing operation after the finger 300 is tilted downward (or backward).

The display device 200 may select an optimal PPG signal (e.g., one having the highest signal quality) among the first PPG signal and the at least one second PPG signal (S180). In some embodiments, a processor of the display device 200 may compare all or a portion of the following: the magnitude of an AC component of the first PPG signal, the magnitude of an AC component of the left tilt PPG signal, the magnitude of an AC component of the right tilt PPG signal, the magnitude of an AC component of the upward tilt PPG signal and the magnitude of an AC component of the downward tilt PPG signal with one another. The processor may then select the optimal PPG signal having the largest AC component among the first PPG signal, the left tilt PPG signal, the right tilt PPG signal, the upward tilt PPG signal and the downward tilt PPG signal. Further, the display device 200 may store optimal guidance image information representing a guidance image corresponding to the optimal PPG signal among the initial guidance image, the left tilt guidance image, the right tilt guidance image, the upward tilt guidance image 230c and the downward tilt guidance image 230d as an optimal guidance image for the user (S185). The display device 200 may determine a biomarker of the user based on the optimal PPG signal, and may display the biomarker (S190).

If the user subsequently requests sensing of the biomarker after the optimal guidance image information is stored (S100: NO), the display device 200 may display the optimal guidance image for the user based on the optimal guidance image information (S192). When the user views the optimal guidance image and places the finger 300 at the optimal position, the display device 200 may generate a PPG signal by performing the PPG sensing operation (S194).

As described above, in the method of providing the biomarker according to embodiments, the first PPG signal may be generated by performing the PPG sensing operation when the finger 300 of the user is located in the sensing region 250, at least one second PPG signal may be generated by performing the PPG sensing operation after at least one tilt guidance image is displayed, the optimal PPG signal may be selected among the first PPG signal 410 and the at least one second PPG signal, and the biomarker of the user may be determined based on the optimal PPG signal. Accordingly, a contact surface between the finger 300 and the sensing region 250 may be optimized for each user, the signal quality of the PPG signal may be improved, and the biomarker of the user may be accurately sensed.

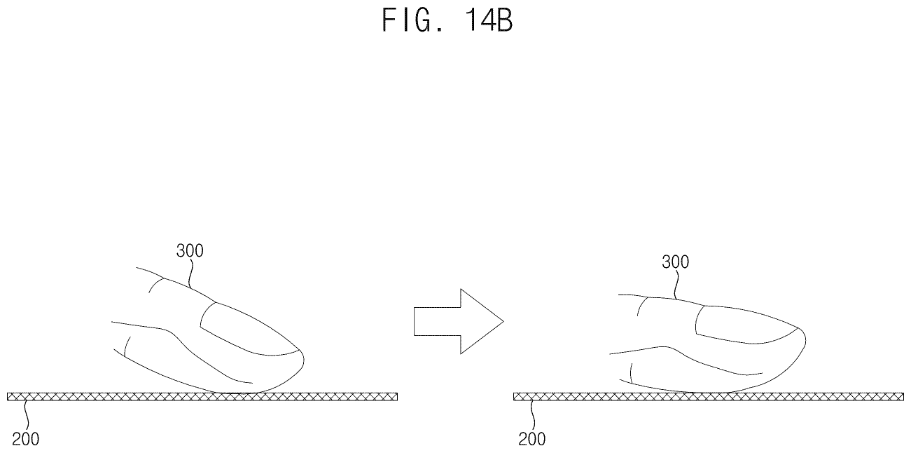

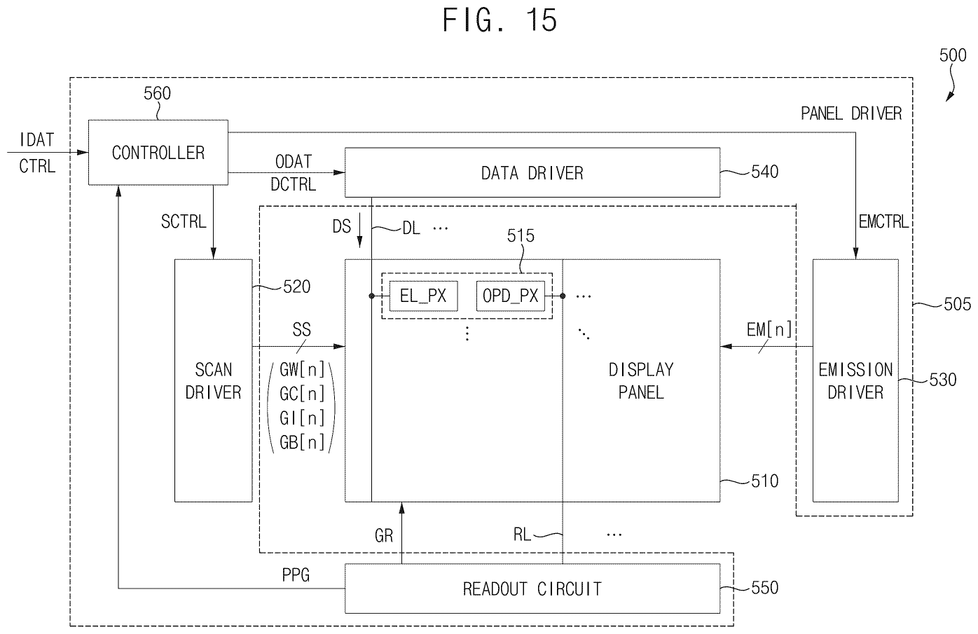

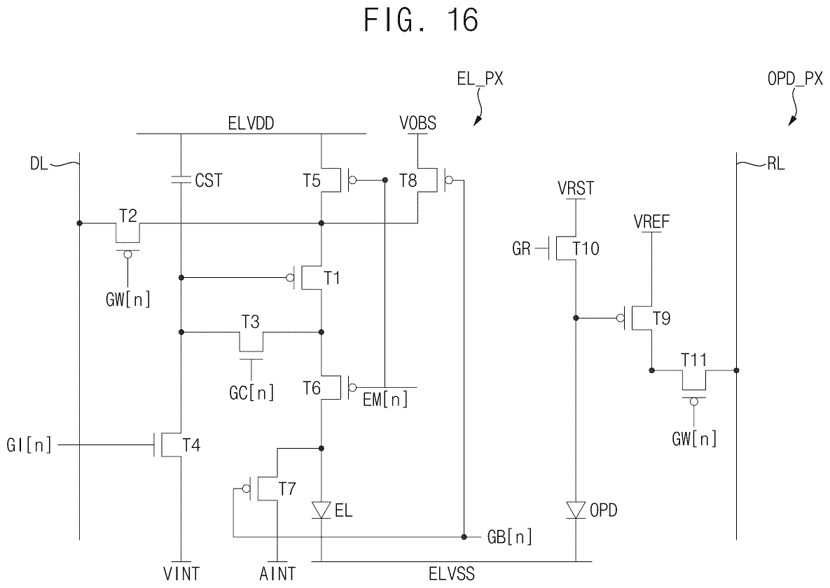

FIG. 15 is a block diagram illustrating a display device according to embodiments, FIG. 16 is a circuit diagram illustrating an example of a light emitting pixel and a light sensing pixel included in a display device according to embodiments, and FIG. 17 is a diagram illustrating an example of a sensing region in which a light sensing pixel is driven and an adjacent region in which a light emitting pixel is driven.

Referring to FIG. 15, a display device 500 according to embodiments may include a display panel 510 that includes a plurality of pixel groups 515, and a panel driver 505 that drives the display panel 510. In some embodiments, as illustrated in FIG. 15, the panel driver 505 may include a scan driver 520 that provides scan signals SS to the display panel 510, an emission driver 530 that provides emission signals EM[n] to the display panel 510, a data driver 540 connected to the display panel 510 through data lines DL, a readout circuit 550 connected to the display panel 510 through readout lines RL, and a controller 560 that controls an operation of the display device 500.

The display panel 510 may include the plurality of pixel groups 515 arranged in a predetermined pattern (e.g., a matrix having a plurality of rows and a plurality of columns). Each pixel group 515 may include at least one light emitting pixel EL_PX having a light emitting element and at least one light sensing pixel OPD_PX having an organic photodiode. Although FIG. 15 illustrates an example in which each pixel group 515 includes one light emitting pixel EL_PX and one light sensing pixel OPD_PX, the number of the light emitting pixel EL_PX and/or the number of the light sensing pixels OPD_PX included in each pixel group 515 are not limited to the example of FIG. 15. For example, in other embodiments, each pixel group 515 may include four light emitting pixels EL_PX (e.g., one red light emitting pixel, two green light emitting pixels and one blue light emitting pixel), and one light sensing pixel OPD_PX.

Each light emitting pixel EL_PX may have a predetermined number of transistors and capacitors. For example, as illustrated in FIG. 16, each light emitting pixel EL_PX may include a first transistor T1 that generates a driving current, a second transistor T2 that transfers a data signal DS of the data line DL in response to a write signal GW[n], a third transistor T3 that diode-connects the first transistor T1 in response to a compensation signal GC[n], a fourth transistor T4 that transfers an initialization voltage VINT to a gate of the first transistor T1 in response to an initialization signal GI[n], a fifth transistor T5 that connects a line of a first power supply voltage ELVDD and the first transistor T1 in response to the emission signal EM[n], a sixth transistor T6 that connects the first transistor T1 and the light emitting element EL in response to the emission signal EM[n], a seventh transistor T7 that transfers an anode initialization voltage AINT to the light emitting element EL in response to a bypass signal GB[n], an eighth transistor T8 that transfers a bias voltage VOBS to a terminal (e.g., a source) of the first transistor T1 in response to the bypass signal GB[n], a storage capacitor CST connected between the line of the first power supply voltage ELVDD and the gate of the first transistor T1, and the light emitting element EL that emits light based on the driving current.

According to embodiments, the light emitting element EL may include an organic light emitting diode (OLED), a nano light emitting diode (NED), a quantum dot (QD) light emitting diode, a micro light emitting diode, an inorganic light emitting diode, or any other suitable light emitting element. In some embodiments, as illustrated in FIG. 16, the first, second, fifth, sixth, seventh and eighth transistors T1, T2, T5, T6, T7 and T8 may be P-type metal-oxide-semiconductor (PMOS) transistors, and the third and fourth transistors T3 and T4 may be N-type metal-oxide-semiconductor (NMOS) transistors, but are not limited thereto.

Further, as illustrated in FIG. 16, the light sensing pixel OPD_PX may include a predetermined number of transistors, e.g., a ninth transistor T9, a tenth transistor T10, an eleventh transistor T11 and the organic photodiode OPD. The ninth transistor T9 may generate a sensing current based on a voltage of an anode of the organic photodiode OPD. In some embodiments, the ninth transistor T9 may include a gate connected to the anode of the organic photodiode OPD, a first terminal that receives a reference voltage VREF, and a second terminal.

The tenth transistor T10 may reset the voltage of the anode of the organic photodiode OPD to a reset voltage VRST in response to a global reset signal GR. In some embodiments, the tenth transistor T10 may include a gate that receives the global reset signal GR, a first terminal that receives the reset voltage VRST, and a second terminal connected to the anode of the organic photodiode OPD.

The eleventh transistor T11 may transfer the sensing current generated by the ninth transistor T9 to the readout line RL in response to the write signal GW[n]. In some embodiments, the eleventh transistor T11 may include a gate that receives the write signal GW[n], a first terminal connected to the second terminal of the ninth transistor T9, and a second terminal connected to the readout line RL.

The organic photodiode OPDs may be used to measure light intensity. For example, after the voltage of the anode of the organic photodiode OPD is reset to the reset voltage VRST, the voltage of the anode of the organic photodiode OPD may be increased by different amounts depending on the light intensity. The sensing current of the ninth transistor T9 may be determined according to the voltage of the anode of the organic photodiode OPD. The readout circuit 550 may generate a PPG signal corresponding to the sensing current. In some embodiments, the organic photodiode OPD may include the anode connected to the gate of the ninth transistor T9, and a cathode connected to a line of a second power supply voltage ELVSS.

For example, when the heart of a user contracts and the volume of a blood vessel in the user's finger increases, the intensity of light reflected from the blood vessel may be decreased, and the voltage of the anode of the organic photodiode OPD may be increased by a relatively small amount. In this case, the ninth transistor T9 may generate a relatively large sensing current based on a relatively low anode voltage, and the readout circuit 550 may generate the PPG signal having a relatively large value based on the relatively large sensing current. For example, the readout circuit 550 may generate the PPG signal having the relatively large value when the volume of the blood vessel increases.

When the heart of the user expands (or relaxes) and the volume of the blood vessel decreases, the intensity of light reflected from the blood vessel may be increased, and the voltage of the anode of the organic photodiode OPD may be increased by a relatively large amount. In this case, the ninth transistor T9 may generate a relatively small sensing current based on a relatively high anode voltage. The readout circuit 550 may generate the PPG signal having a relatively small value based on the relatively small sensing current. For example, the readout circuit 550 may generate the PPG signal having the relatively small value when the volume of the blood vessel decreases. Accordingly, the PPG signal may have a value corresponding to the volume of the blood vessel.

In some embodiments, as illustrated in FIG. 16, the ninth and eleventh transistors T9 and T11 may be PMOS transistors, and the tenth transistor T10 may be an NMOS transistor, but are not limited thereto. Additionally, although FIG. 16 illustrates an example of the light emitting pixel EL_PX and the light sensing pixel OPD_PX, the light emitting pixel EL_PX and the light sensing pixel OPD_PX of the display device 500 according to embodiments are not limited to the example of FIG. 16, and may have any structure.

The scan driver 520 may generate the scan signals SS based on a scan control signal SCTRL received from the controller 560, and may sequentially provide the scan signals SS to the display panel 510 on a row-by-row basis. In some embodiments, the scan control signal SCTRL may include, but is not limited to, a scan start signal and a scan clock signal. Further, the scan signal SS may include, but is not limited to, the write signal GW[n], the compensation signal GC[n], the initialization signal GI[n] and the bypass signal GB[n]. Further, in some embodiments, the scan driver 520 may be integrated or formed in the display panel 510. In other embodiments, the scan driver 520 may be implemented as one or more integrated circuits.

The emission driver 530 may generate the emission signals EM[n] based on an emission control signal EMCTRL received from the controller 560, and may sequentially provide the emission signals EM[n] to the display panel 510 on a row-by-row basis. In some embodiments, the emission control signal EMCTRL may include, but is not limited to, an emission start signal and an emission clock signal. Further, in some embodiments, the emission driver 530 may be integrated or formed in the display panel 510. In other embodiments, the emission driver 530 may be implemented as one or more integrated circuits.

The data driver 540 may generate data signals DS based on a data control signal DCTRL and output image data ODAT received from the controller 560, and may provide the data signals DS to the light emitting pixels EL_PX through the data lines DL. In some embodiments, the data control signal DCTRL may include, but is not limited to, an output data enable signal, a horizontal start signal, and a load signal. In some embodiments, the data driver 540 and the controller 560 may be implemented as a single integrated circuit, and the single integrated circuit may be referred to as a timing controller embedded data driver (TED) integrated circuit. In other embodiments, the data driver 540 and the controller 560 may be implemented as separate integrated circuits.

The readout circuit 550 may receive the sensing currents of the light sensing pixels OPD_PX through the readout lines RL, may generate a PPG signal based on the sensing currents, and may provide the PPG signal to the controller 560. Further, the readout circuit 550 may substantially simultaneously apply the global reset signal GR to all the light sensing pixels OPD_PX of the display panel 510. In some embodiments, the readout circuit 550 may be implemented as an integrated circuit, and the integrated circuit may be referred to as a readout integrated circuit (ROIC). In other embodiments, the readout circuit 550 may be included in the data driver 540.

The controller 560 (e.g., a timing controller (TCON)) may receive input image data IDAT and a control signal CTRL from an external host processor (e.g., a graphics processing unit (GPU), an application processor (AP), or a graphics card). In some embodiments, the input image data IDAT may be RGB image data including red image data, green image data and blue image data. In some embodiments, the control signal CTRL may include, but is not limited to, a vertical synchronization signal, a horizontal synchronization signal, an input data enable signal, and a master clock signal. The controller 560 may generate the output image data ODAT, the data control signal DCTRL, the scan control signal SCTRL and the emission control signal EMCTRL based on the input image data IDAT and the control signal CTRL. The controller 560 may control an operation of the data driver 540 by providing the output image data ODAT and the data control signal DCTRL to the data driver 540, may control an operation of the scan driver 520 by providing the scan control signal SCTRL to the scan driver 520, and may control an operation of the emission driver 530 by providing the emission control signal EMCTRL to the emission driver 530.

In the display device 500 according to embodiments, a processor (e.g. a panel driver) may drive the display panel 510 to display an initial guidance image that guides the user to place a finger on a sensing region of the display panel 510. When the finger is located in the sensing region, the panel driver 505 may perform a PPG sensing operation to generate a first PPG signal. For example, as illustrated in FIG. 17, to perform the PPG sensing operation, the panel driver 505 may drive the light emitting pixels EL_PX of the pixel groups 515 located in an adjacent region 580 adjacent to a sensing region 570 (e.g., the adjacent region 580 surrounding the sensing region 570) to emit light, and may drive the light sensing pixels OPD_PX of the pixel groups 515 located in the sensing region 570 to sense the light reflected from the blood vessel of the finger of a user. Further, the readout circuit 550 may generate the first PPG signal based on the sensing currents of the optical sensing pixels OPD_PX within the sensing region 570.

Thereafter, the panel driver 505 may drive the display panel 510 to display at least one tilt guidance image that requests to tilt the finger in at least one direction. When the finger is tilted in the at least one direction, the panel driver 505 may generate at least one second PPG signal by performing the PPG sensing operation. Further, the panel driver 505 may select an optimal PPG signal having the highest signal quality among the first PPG signal and the at least one second PPG signal, may determine a biomarker of the user based on the optimal PPG signal, and may drive the display panel 510 to display the biomarker of the user.

Further, the panel driver 505 may store optimal guidance image information representing a guidance image corresponding to the optimal PPG signal among the initial guidance image and the at least one tilt guidance image as an optimal guidance image for the user. If the user subsequently requests sensing of the biomarker, the panel driver 505 may drive the display panel 510 to display the optimal guidance image for the user based on the optimal guidance image information. Accordingly, the contact surface between the finger and the sensing region 570 may be optimized for each particular user, signal quality of the PPG signal may be improved, and the biomarker of the user may be accurately sensed.

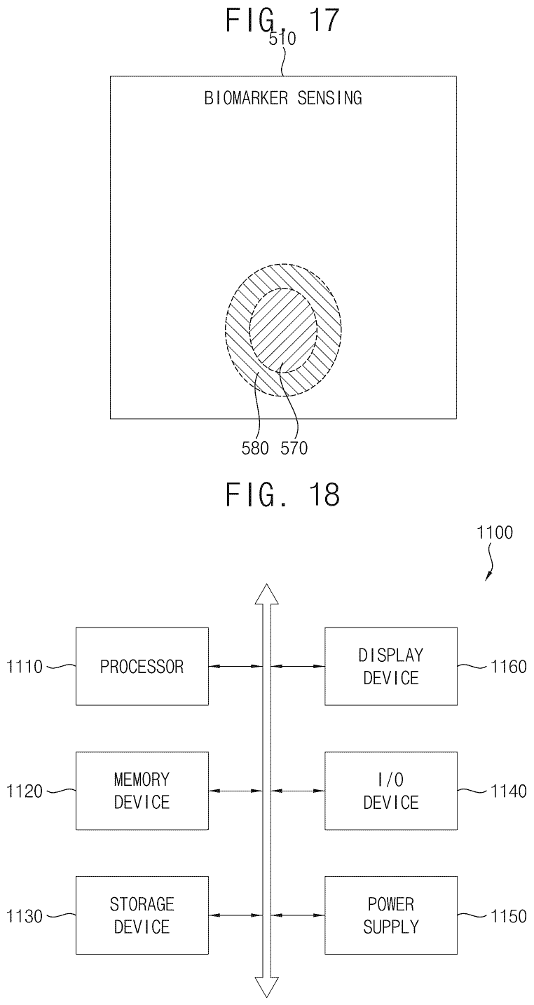

FIG. 18 is a block diagram illustrating an electronic device 1100 including a display device according to embodiments.

Referring to FIG. 18, the electronic device 1100 may include a processor 1110, a memory device 1120, a storage device 1130, an input/output (I/O) device 1140, a power supply 1150, and a display device 1160. The electronic device 1100 may further include a plurality of ports for communicating with a video card, a sound card, a memory card, a universal serial bus (USB) device, other electronic devices, etc.

The processor 1110 may perform various computing functions or tasks. The processor 1110 may be an application processor (AP), a micro-processor, a central processing unit (CPU), etc. The processor 1110 may be coupled to other components via an address bus, a control bus, a data bus, etc. Further, in some embodiments, the processor 1110 may be further coupled to an extended bus such as a peripheral component interconnection (PCI) bus. In one embodiment, the processor 1110 may correspond to the panel driver previously mentioned.

The memory device 1120 may store data for operations of the electronic device 1100. For example, the memory device 1120 may include at least one non-volatile memory device such as an erasable programmable read-only memory (EPROM) device, an electrically erasable programmable read-only memory (EEPROM) device, a flash memory device, a phase change random access memory (PRAM) device, a resistance random access memory (RRAM) device, a nano floating gate memory (NFGM) device, a polymer random access memory (PoRAM) device, a magnetic random access memory (MRAM) device, a ferroelectric random access memory (FRAM) device, etc., and/or at least one volatile memory device such as a dynamic random access memory (DRAM) device, a static random access memory (SRAM) device, a mobile dynamic random access memory (mobile DRAM) device, etc. In one embodiment, the memory device 1120 may store the guidance images and image information previously discussed.

The storage device 1130 may be a solid state drive (SSD) device, a hard disk drive (HDD) device, a CD-ROM device, etc. The I/O device 1140 may be an input device such as a keyboard, a keypad, a mouse, a touch screen, etc., and an output device such as a printer, a speaker, etc. The power supply 1150 may supply power for operations of the electronic device 1100. The display device 1160 may be coupled to other components through the buses or other communication links.

In the display device 1160, a first PPG signal may be generated by performing a PPG sensing operation when a finger of a user is located in a sensing region of the display device 1160, at least one second PPG signal may be generated by performing the PPG sensing operation after at least one tilt guidance image is displayed, an optimal PPG signal may be selected among the first PPG signal and the at least one second PPG signal, and a biomarker of the user may be determined based on the optimal PPG signal. Accordingly, a contact surface between the finger and the sensing region may be optimized for each user, a signal quality of a PPG signal may be improved, and the biomarker of the user may be accurately sensed.

The inventive concepts may be applied to any electronic device 1100 including the display device 1160. For example, the inventive concepts may be applied to a smart phone, a wearable electronic device, a tablet computer, a mobile phone, a television (TV) (e.g., a digital TV or a 3D TV), a personal computer (PC), a home appliance, a laptop computer, a personal digital assistant (PDA), a portable multimedia player (PMP), a digital camera, a music player, a portable game console, a navigation device, etc.

In one or more embodiments, the guidance images (e.g., right tilt, left tilt, upward tilt, downward tilt, upward movement, downward movement, etc.) described herein may be generally referred to as finger adjustment images. The finger adjustment images may include any of the tilt or movement guidance images previously discussed, and corresponding PPG signals may be generated and compared to determine an optimal PPG signal.

The methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

Also, another embodiment may include a computer-readable medium, e.g., a non-transitory computer-readable medium, for storing the code or instructions described above. The computer-readable medium may be a volatile or non-volatile memory or other storage device, which may be removably or fixedly coupled to the computer, processor, controller, or other signal processing device which is to execute the code or instructions for performing the method embodiments or operations of the apparatus embodiments herein.

The controllers, processors, drivers, circuits, and other signal generating and signal processing features of the embodiments disclosed herein may be implemented, for example, in non-transitory logic that may include hardware, software, or both. When implemented at least partially in hardware, the controllers, processors, drivers, circuits, and other signal generating and signal processing features may be, for example, any one of a variety of integrated circuits including but not limited to an application-specific integrated circuit, a field-programmable gate array, a combination of logic gates, a system-on-chip, a microprocessor, or another type of processing or control circuit. In some embodiments, these features may be implemented by a neural network, machine-learning logic, or other form of artificial intelligence. \

When implemented in at least partially in software, the controllers, processors, drivers, circuits, and other signal generating and signal processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein

The foregoing is illustrative of embodiments and is not to be construed as limiting thereof. Although a few embodiments have been described, those skilled in the art will readily appreciate that many modifications are possible in the embodiments without materially departing from the novel teachings and advantages of the present inventive concept. Accordingly, all such modifications are intended to be included within the scope of the present inventive concept as defined in the claims. Therefore, it is to be understood that the foregoing is illustrative of various embodiments and is not to be construed as limited to the specific embodiments disclosed, and that modifications to the disclosed embodiments, as well as other embodiments, are intended to be included within the scope of the appended claims. The embodiments may be combined to form additional embodiments.

What is claimed is:

1. A method of providing a biomarker by a display device, the method comprising:
displaying an initial guidance image that guides a user to place a finger on a sensing region of the display device;
generating a first photoplethysmography (PPG) signal by performing a PPG sensing operation when the finger is located on the sensing region;
displaying at least one tilt guidance image that requests the user to tilt the finger in at least one direction;
generating at least one second PPG signal by performing the PPG sensing operation when the finger is tilted in the at least one direction on the sensing region;
selecting an optimal PPG signal having a highest signal quality among the first PPG signal and the at least one second PPG signal; and
displaying a biomarker of the user determined based on the optimal PPG signal.

2. The method of claim 1, wherein:
a display panel of the display device includes a light emitting pixel having a light emitting element and a light sensing pixel having an organic photodiode, and
the PPG sensing operation is performed such that the light emitting pixel emits light and the light sensing pixel senses the light reflected from a blood vessel of the finger.

3. The method of claim 1, wherein displaying the at least one tilt guidance image includes:
displaying a left tilt guidance image that requests the user to tilt the finger to left; and
displaying a right tilt guidance image that requests the user to tilt the finger to right.

4. The method of claim 3, wherein generating the at least one second PPG signal includes:
generating a left tilt PPG signal by performing the PPG sensing operation when the finger is tilted to the left; and
generating a right tilt PPG signal by performing the PPG sensing operation when the finger is tilted to the right.

5. The method of claim 4, wherein selecting the optimal PPG signal includes:
comparing a magnitude of an alternate current (AC) component of the first PPG signal, a magnitude of an AC component of the left tilt PPG signal and a magnitude of an AC component of the right tilt PPG signal; and
selecting the optimal PPG signal having a largest AC component among the first PPG signal, the left tilt PPG signal and the right tilt PPG signal.

6. The method of claim 3, wherein displaying the at least one tilt guidance image further includes:
displaying an upward tilt guidance image that requests to tilt the finger upward; and
displaying a downward tilt guidance image that requests to tilt the finger downward.

7. The method of claim 6, wherein generating the at least one second PPG signal includes:
generating a left tilt PPG signal by performing the PPG sensing operation when the finger is tilted to the left;
generating a right tilt PPG signal by performing the PPG sensing operation when the finger is tilted to the right;
generating an upward tilt PPG signal by performing the PPG sensing operation when the finger is tilted upward; and
generating a downward tilt PPG signal by performing the PPG sensing operation when the finger is tilted downward.

8. The method of claim 7, wherein selecting the optimal PPG signal includes:
comparing a magnitude of an AC component of the first PPG signal, a magnitude of an AC component of the left tilt PPG signal, a magnitude of an AC component of the right tilt PPG signal, a magnitude of an AC component of the upward tilt PPG signal and a magnitude of an AC component of the downward tilt PPG signal; and
selecting the optimal PPG signal having a largest AC component among the first PPG signal, the left tilt PPG signal, the right tilt PPG signal, the upward tilt PPG signal and the downward tilt PPG signal.

9. The method of claim 1, further comprising:
displaying at least one movement guidance image that requests movement of the finger; and
generating at least one third PPG signal by performing the PPG sensing operation when the finger moves.

10. The method of claim 9, wherein displaying the at least one tilt guidance image includes:
displaying a left tilt guidance image that requests to tilt the finger to left; and
displaying a right tilt guidance image that requests to tilt the finger to right, and wherein displaying the at least one movement guidance image includes:
displaying an upward movement guidance image that requests the user to move the finger upward; and
displaying a downward movement guidance image that requests the user to move the finger downward.

11. The method of claim 10, wherein generating the at least one second PPG signal includes:
generating a left tilt PPG signal by performing the PPG sensing operation when the finger is tilted to the left; and
generating a right tilt PPG signal by performing the PPG sensing operation when the finger is tilted to the right, and wherein generating the at least one third PPG signal includes:
generating an upward movement PPG signal by performing the PPG sensing operation when the finger moves upward; and
generating a downward movement PPG signal by performing the PPG sensing operation when the finger moves downward.

12. The method of claim 11, wherein selecting the optimal PPG signal includes:
comparing a magnitude of an AC component of the first PPG signal, a magnitude of an AC component of the left tilt PPG signal, a magnitude of an AC component of the right tilt PPG signal, a magnitude of an AC component of the upward movement PPG signal and a magnitude of an AC component of the downward movement PPG signal; and
selecting the optimal PPG signal having a largest AC component among the first PPG signal, the left tilt PPG signal, the right tilt PPG signal, the upward movement PPG signal and the downward movement PPG signal.

13. The method of claim 1, wherein displaying the biomarker of the user includes:
displaying at least one of a blood pressure, a heart rate, a stress level, or a cardiovascular health of the user determined based on the optimal PPG signal.

14. The method of claim 1, further comprising:
storing optimal guidance image information representing an optimal guidance image for the user, wherein the optimal guidance image is a guidance image corresponding to the optimal PPG signal among the initial guidance image and the at least one tilt guidance image.

15. The method of claim 14, further comprising:
displaying the optimal guidance image for the user based on the optimal guidance image information when the user subsequently requests sensing of the biomarker;
generating a subsequent PPG signal by performing the PPG sensing operation; and
displaying the biomarker of the user determined based on the subsequent PPG signal.

16. A display device comprising:
a display panel including a plurality of pixel groups, each of the pixel groups including a light emitting pixel having a light emitting element and a light sensing pixel having an organic photodiode; and
a panel driver configured to drive the display panel,
wherein the panel driver is configured to:
drive the display panel to display an initial guidance image that guides a user to place a finger on a sensing region of the display panel,
generate a first photoplethysmography (PPG) signal by performing a PPG sensing operation when the finger is located on the sensing region;
drive the display panel to display at least one tilt guidance image that requests the user to tilt the finger in at least one direction;
generate at least one second PPG signal by performing the PPG sensing operation when the finger is tilted in the at least one direction;
select an optimal PPG signal having a highest signal quality among the first PPG signal and the at least one second PPG signal; and
drive the display panel to display a biomarker of the user determined based on the optimal PPG signal.

17. A method of providing a biomarker by a display device, the method comprising:
generating a first biological signal based on a sensing operation performed for a finger of a user on a display screen;
displaying a finger adjustment image instructing the user to adjust a position of the finger on the display screen;
generating a second biological signal based on a sensing operation performed for the adjusted position of the finger;
selecting the first biological signal or the second biological signal; and
displaying a biomarker on the display screen based on the selected first biological signal or the second biological signal.

18. The method of claim 17, wherein each of the first biological signal and the second biological signal is a photoplethysmography (PPG) signal.

19. The method of claim 17, wherein selecting the first biological signal or the second biological signal includes:
determining a signal quality of the first biological signal;
determining a signal quality of the second biological signal;
comparing the signal quality of the first biological signal and the signal quality of the second biological signal; and
selecting the second biological signal based on the comparison, and wherein the second PPG signal has a higher signal quality than the first PPG signal.

20. The method of claim 17, whether the finger adjustment image instructs the user to tilt or move the finger.
